(19) United States
(12) Patent Application Publication
(10) Pub. No.: US 2026/0174666 A1
(43) Pub. Date: Jun. 25, 2026

(54) DRY SHAMPOO WITH INCREASED SOLVENT CONTENT

(71) Applicant: Church & Dwight Co., Inc., Princeton, NJ (US)

(72) Inventors: Gabrielle BARTON, Folkestone (GB); Sophie WALSH, Hythe (GB); Shirish DESALE, Ashford (GB); Alexander RICHARDSON, Chatham (GB)

(73) Assignee: Church & Dwight Co., Inc., Princeton, NJ (US)

(21) Appl. No.: 19/423,155

(22) Filed: Dec. 17, 2025

Related U.S. Application Data
(60) Provisional application No. 63/736,224, filed on Dec. 19, 2024.

(51) Int. Cl. A61K 8/73; A61K 8/04; A61K 8/19; A61K 8/58; A61Q 5/02

(52) U.S. Cl. CPC A61K 8/732; A61K 8/046; A61K 8/19; A61K 8/585; A61Q 5/02

(57) ABSTRACT

The present disclosure provides a method for cleaning hair and a hair cleaning composition that includes a particulate cleaning agent in a total amount of about 2% to about 20% by weight based on a total weight of the hair cleaning composition, a plurality of solvents including at least one water-soluble or water-miscible solvent and at least one solvent that is not water-soluble or water-miscible, and a propellant including nitrogen gas. The plurality of solvents may be present in a total amount of about 70% to about 90% by weight based on the total weight of the hair cleaning composition. The propellant may be present in an amount of about 1% to about 5% by weight based on the total weight of the hair cleaning composition. The hair cleaning composition leaves hair wet upon application to the hair and dries within a time of less than 30 seconds.

### CROSS-REFERENCE TO RELATED APPLICATIONS

This application claims priority to and the benefit of U.S. provisional patent application No. 63/736,224, filed Dec. 19, 2024 and entitled “Dry Shampoo With Increased Solvent Content,” the entire contents of which are incorporated herein by reference.

### FIELD OF THE DISCLOSURE

The present disclosure relates generally to hair cleaning compositions, and more particularly, to hair cleaning compositions that are delivered in pressurized aerosol containers and use compressed gas as the propellant.

### BACKGROUND

Hair cleaning compositions may be either for use on wet hair or on dry hair. Dry shampoos are popular formulations for cleaning hair that are distinct from traditional lathering shampoos. Traditional shampoos require water to create a lather, which is applied to wet hair and rinsed with water to remove the product and oils from hair. Dry shampoo may be applied to dry hair and does not require water or rinsing to absorb oil from hair. Often times, dry shampoo formulations are provided in the form of a composition that is delivered in an aerosol container as a spray. These types of hair care products are commonly packaged in pressurized aerosol containers with a release valve to dispense the pressurized product into the air as an aerosol. Typically, the composition is applied to the hair by spraying the composition onto the scalp or roots of the hair, massaged-in, or combed or brushed through the hair. This is a process that can be done very quickly due to the no-rinse formulation.

Typical dry shampoo products comprise a pressurized container, a nozzle, a propellant, and a hair care composition. In use, the hair care composition is dispensed from the pressurized container by an aerosol-forming nozzle aided by the propellant. Current dry shampoo products use a propellant such as mixture of hydrocarbons including butane, isobutane and propane. Such propellants are often referred to as liquefied petroleum gases (“LPGs”). Although LPGs work very well in the formulation and gives a constant pressure throughout the usage of the product, LPGs are also known as volatile organic compounds (VOCs). VOCs have been implicated in a range of health issues including eye, nose, and throat irritation, headaches, dizziness, damage to the liver, kidneys, and central nervous system. VOCs are often found in higher concentrations indoors than outdoors leading to poor air quality, which can exacerbate respiratory conditions like asthma and allergies. Therefore, using VOCs is not considered a sustainable option, and it remains desirable to reduce the use of products that emit VOCs and opt for more environmentally-friendly alternatives can help mitigate these risks.

A compressed gas, such as nitrogen gas, may be used as an alternative to LPGs as a propellant. It can be difficult to make a consumer acceptable aerosol hairspray product that uses a compressed gas as the propellant because, unlike LPGs, compressed gas is always in the vapor state and therefore the pressure in the container is reduced as product is dispensed, making it difficult to dispense the hairspray composition at a consumer acceptable particle size distribution and spray rate over the life of the container. Compressed gas will not yield a constant pressure and discharge rate throughout the usage of the can, with the pressure slowly decreasing throughout the usage of the can. Eventually, the pressure can be so low that no product is released at all, even if there is product left in the can.

In addition, compressed gas requires a higher level of liquid versus the traditional dry shampoo products using LPGs as a propellant. LPGs as a propellant, unlike nitrogen, is liquified under pressure inside the aerosol and flashes off quickly once expelled from the can. The liquified propellant also provides an efficient carrier for rice starch. When using compressed gas as a propellant, the compressed gas remains in the gaseous state under pressure. A higher level of liquid is required to carry the powder out of the can. Typically, an alcohol, such an ethanol, may be used. This is another VOC that is typically used as the main solvent in aerosol dry shampoos. If the formula has a solvent that is entirely ethanol and uses a high amount to be the carrier for the absorbent powder, the overall levels of VOC are too high to comply with regulations such as the California Air Resources Board (CARB) regulations that regulate the amount of VOCs in consumer products.

Thus, there remains a need for an aerosol product with compressed gas as the propellant while maintaining dispensing performance and longevity while complying with regulations related to the solvent.

### SUMMARY OF THE DISCLOSURE

The present disclosure is directed to a hair cleaning composition that uses a compressed gas as a propellant and a plurality of solvents. The hair cleaning composition may be configured to be applied to hair as an aerosol spray.

In one or more embodiments, the present disclosure can relate to a hair cleaning composition that includes a particulate cleaning agent, a plurality of solvents, and a propellant.

The particulate cleaning agent may be present in the hair cleaning composition in a total amount of about 2% to about 20% by weight based on a total weight of the hair cleaning composition. The particulate cleaning agent may be present in an amount of about 8% to about 15% by weight based on the total weight of the hair cleaning composition. In other embodiments, the particulate cleaning agent may be present in a lower amount of about 5% to about 15% by weight based on a total weight of the hair cleaning composition, or from about 10% to about 15% by weight based on a total weight of the hair cleaning composition. The particulate cleaning agent may include a starch such as a rice starch.

The plurality of solvents may include at least one water-soluble or water-miscible solvent and at least one solvent that is not water-soluble or water-miscible. The plurality of solvents may be present in a total amount of about 70% to about 90% by weight, about 75% to about 85% by weight, or about 80% to about 90% by weight based on the total weight of the hair cleaning composition. The at least one water-soluble or water-miscible solvent may include an alcohol, water, or a mixture of alcohol and water. The at least one solvent that is not water-soluble or water-miscible may include a siloxane such as disiloxane and may include hexamethyldisiloxane.

The propellant may include compressed gas, such as nitrogen gas, and may be present in an amount of about 1% to about 5% by weight, or about 2.5% to about 3.5% by weight based on the total weight of the hair cleaning composition.

The hair cleaning composition may leave hair wet upon application to the hair and dries within a time of less than 30 seconds.

In some embodiments, the plurality of solvents comprises an alcohol, water, and a siloxane.

In some embodiments, the plurality of solvents comprises an alcohol and a siloxane.

In some embodiments, the hair cleaning composition may also include one or more materials such as fragrances, anti-caking agents, conditioning agents, surfactants, preservatives, colorants, natural oils, or synthetic oils, and combinations thereof.

In one or more embodiments, the present disclosure can relate to a method for cleaning hair including the steps of spraying the hair with a hair cleaning composition delivered as an aerosol where the hair is wetted, and mechanically dispersing the hair cleaning composition through the hair such that one or both of oil and grease on the hair is at least partially removed by the particulate cleaning agent and the wetted hair is substantially dry within a time of less than 30 seconds, about 2 seconds to about 20 seconds, or about 5 seconds to about 15 seconds. The hair cleaning composition may include a particulate cleaning agent; a plurality of solvents; and a propellant consisting essentially of compressed gas, such as nitrogen.

In some embodiments, the step of mechanically dispersing the hair cleaning composition through the hair includes brushing or combing the hair, or running of fingers through the hair.

Aspects and advantages of these exemplary embodiments and other embodiments, are discussed in detail herein. Moreover, it is to be understood that both the foregoing information and the following detailed description provide merely illustrative examples of various aspects and embodiments and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### DETAILED DESCRIPTION OF THE DISCLOSURE

The invention now will be described more fully herein after through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” are defined as being close to as understood by one of ordinary skill in the art. In one non-limiting embodiment, the terms are defined to be within 10%, preferably within 5%, more preferably within 1%, and most preferably within 0.5%. The terms “removing,” “removed,” “reducing,” “reduced,” or any variation thereof, when used in the claims and/or the specification includes any measurable decrease of one or more components in a mixture to achieve a desired result. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The present disclosure relates to a hair cleaning composition that includes a particulate cleaning agent, a plurality of solvents; and a propellant, where the hair cleaning composition has a wet-to-dry transition by leaving hair wet upon application and drying within a time of less than 30 seconds.

Hair cleaning compositions, such as dry shampoos, are often delivered in pressurized aerosol cans that use hydrocarbons as the propellant. Hydrocarbons like Liquefied Petroleum Gas (LPG), which includes butane, propane, and isobutane, are commonly used as propellants in aerosol dry shampoos. LPGs maintain a consistent pressure inside aerosol cans. As the hair cleaning compositions is used, the LPG evaporates to replace the gas in the can, ensuring a steady spray performance throughout the can's life. LPGs are also relatively inexpensive compared to other propellants, which makes them a cost-effective choice for manufacturers. LPGs are also stable compounds, which helps in maintaining the quality and effectiveness of the product on the shelf. LPGs offer a wide range of vapor pressures and boiling points, making them suitable for various applications.

There are several reasons why the use of LPGs may be undesirable. LPGs can pose significant health risks. When a user sprays dry shampoo, gases may be released into the air and can be inhaled. LPGs are highly flammable, and using products containing LPGs near heat sources or open flames may be dangerous.

Using compressed gases, such as nitrogen, as an alternative to LPGs in aerosol products is desirable but presents several challenges. Unlike LPGs, which are liquefied under pressure and provide consistent pressure throughout usage, compressed gases, such as nitrogen, remain in a vapor state at ambient temperature. This results in a gradual decrease in pressure as the product is dispensed, leading to inconsistent spray performance, and eventually making it difficult to release any product at all. Additionally, compressed gases, such as nitrogen, require a higher liquid content to effectively carry the product out of the can. For example, ethanol is often used as a solvent in aerosol dry shampoos. If a compressed gas, such as nitrogen, is used as the propellant, the amount of ethanol must be increased accordingly. However, using high amounts of ethanol can lead to excessive levels of volatile organic compounds (VOCs), which may not comply with environmental regulations like those set by the California Air Resources Board (CARB).

Therefore, there is a need for an aerosol product that uses non-LPG compressed gas as a propellant while maintaining consistent performance and adhering to regulatory standards.

In one or more embodiments, the present disclosure provides a hair cleaning composition that uses a compressed gas, such as nitrogen, in combination with a plurality of solvents and a cleaning agent. The hair cleaning composition dries quickly, absorbs oil efficiently, and results in a refreshing feel on the hair.

The hair cleaning composition contains a mixture or plurality of solvents rather than only alcohol so that the product complies with environmental regulations and standards. The hair cleaning composition includes a plurality of solvents including at least one water-soluble or water-miscible solvent and at least one solvent that is not water-soluble or water-miscible. The water-soluble or water-miscible solvent may include an alcohol, water, or a mixture of the alcohol and water. The solvent that is not water-soluble or water-miscible may include a siloxane. The plurality of solvents may include an alcohol, water, and a siloxane. The plurality of solvents may include an alcohol and a siloxane. The alcohol may include ethanol. The siloxane may include a disiloxane, which may specifically include hexamethyldisiloxane (HMDSO).

The mixture of solvents including alcohol and water works surprisingly well to provide a refreshing feel on the head and hair without causing the cleaning agent or powder to cake or block the nozzle of the can.

The mixture of solvents including ethanol and hexamethyldisiloxane surprisingly does not make the hair feel greasy and the volatility of the hexamethyldisiloxane allows the product to easily disperse.

The plurality of solvents may be present in a total amount of about 70% to about 90% by weight, about 72% to about 88% by weight, about 74% to about 86% by weight, about 76% to about 84% by weight, about 78% to about 82% by weight, about 75% to about 85% by weight, or about 80% to about 90% by weight, based on the total weight of the hair cleaning composition.

The hair cleaning composition may also include a particulate cleaning agent. In the context of a dry shampoo product, a particulate cleaning agent is typically a fine powder that helps absorb excess oil and grease from hair and the scalp, remove moisture, add texture, and/or add volume. The particulate cleaning agent may include tapioca starch, rice starch, corn starch, kaolin clay, silica, or any combination thereof. These ingredients work by adhering to the oils and impurities in the hair, making it easier to brush, comb, or shake out, leaving the hair looking and feeling cleaner or refreshed.

The particulate cleaning agent may be present in the hair cleaning composition in a total amount of about 10% to about 30% by weight, about 10% to about 20% by weight, about 12% to about 28% by weight, about 14% to about 26% by weight, about 15% to about 25% by weight, about 16% to about 24% by weight, or about 18% to about 22% by weight, based on a total weight of the hair cleaning composition. In other embodiments, the particulate cleaning agent may be present in the hair cleaning composition in a lower amount of 2% to about 20% by weight, about 4% to about 18% by weight, about 6% to about 16% by weight, about 8% to about 14% by weight, about 10% to about 12% by weight, about 4% to about 16% by weight, about 8% to about 15% by weight, or about 6% to about 12% by weight, based on a total weight of the hair cleaning composition. A lower amount of particulate cleaning agent may prevent or minimize the visible residue or build-up left on the hair and scalp, making it more suitable for people with darker hair colors, as it reduces the risk of a white or chalky appearance. A lower amount of particulate cleaning agent may also result in smoother application and feel, avoiding a gritty or heavy texture, which can be more comfortable for the user and less likely to weigh the hair down. A lower amount of particulate cleaning agent may also prevent or reduce nozzle blockages in the aerosol product.

The hair cleaning composition may also include a propellant. In an aerosol product, such as an aerosol can, a propellant is responsible for creating the pressure needed to dispense the product from the can. Common propellants include hydrocarbons, including LPGs like ethane, propane, butane, isopentane, pentane, and isobutane. These gases are compressed inside the can, and when the nozzle of the can is depressed during usage, the propellant forces the product, such as a hair cleaning composition, out of the can in a fine mist, allowing for even application. Hydrocarbons are often chosen for their ability to be easily liquefied under pressure and are effective in creating a fine mist when dispensed.

In the present disclosure, the propellant may include a compressed gas rather than a common propellant such as LPGs. Compressed gases may include nitrogen, carbon dioxide, and air. Compressed gases are non-flammable and environmentally friendly compared to other propellants like hydrocarbons. Compressed gases are stored under high pressure inside the can. When the nozzle is depressed, the compressed gas forces the product out in a fine mist or spray for even application.

In some embodiments, the propellant may only include or specifically include nitrogen. The propellant may consist of or consist essentially of nitrogen. The phrase “consisting essentially of” encompasses propellants that remain in gaseous form when compressed in an aerosol or spray container.

The propellant may be present in the hair cleaning composition in an amount of about 1% to about 5% by weight, about 1.5% to about 4.5% by weight, about 2% to about 4% by weight, or about 2.5% to about 3.5% by weight, based on the total weight of the hair cleaning composition.

In some embodiments, the hair cleaning composition may include additional components or materials such as fragrances, anti-caking agents, conditioning agents, surfactants, preservatives, colorants, natural oils, or synthetic oils, and combinations thereof.

Fragrance may be included in the hair cleaning composition. The fragrance may give hair a fresh, clean scent to mimic a recent washing. The fragrance may include floral scents, citrus scents, herbal scents, or fruity scents. The amount of fragrance present in the hair cleaning composition may range from about 0.1% to about 1%, about 0.2% to about 0.9%, about 0.3% to about 0.7%, about 0.4% to about 0.6%, about 0.1% to about 0.5%, or about 0.5% to about 1%.

Anti-caking agents prevent the powder from clumping together by absorbing moisture and ensuring the product remains free-flowing. The anti-caking agents may include cetrimonium chloride.

Conditioning agents are designed to improve the feel, appearance, and manageability of hair and scalp. The conditioning agents may include distearyldimonium chloride, silicones, emollients, humectants, and proteins.

Surfactants may also be used in the hair cleaning compositions to cleanse and remove impurities, oil, and product buildup from the hair and scalp. The surfactants may include amphoteric surfactants such as cocamidopropyl betaine and lauryl betaine.

In some embodiments, the present disclosure may also include a method of cleaning hair. The hair cleaning composition may be configured to be applied to hair as an aerosol spray. The method may include the steps of spraying the hair with a hair cleaning composition delivered as an aerosol where the hair is wetted. The term “wetted” means that liquid components in the hair cleaning composition remain adhered to surfaces of the hair so that the hair is one or both of visibly wet and touchably wet (i.e., feels wet to touch, such as upon contact with skin). Wetting the hair can provide a sensation to a user that is much closer to the experience of a traditional hair wash where hair is wetted with water, washed with soap or shampoo, and the soap or shampoo is rinsed from the hair with water, removing dirt and oils from the hair.

The method may also include a step of mechanically dispersing the hair cleaning composition through the hair such that one or both of oil and grease on the hair is at least partially removed by the particulate cleaning agent. The step of mechanically dispersing the hair cleaning composition through the hair may include brushing or combing the hair with an implement, running fingers through the hair, or shaking the hair vigorously with fingers or hands.

The presence of the mixture or plurality of solvents in the hair cleaning composition results in a wet application of the product on the hair that dries quickly to result in refreshed and clean hair. The wetted hair is substantially dry within a time of less than 30 seconds, about 2 seconds to about 20 seconds, about 5 seconds to about 25 seconds, about 5 seconds to about 15 seconds, about 10 seconds to about 20 seconds, or about 10 seconds to about 15 seconds. This is a sharp contrast to traditional hair washing with water where hair can take minutes or even hours to dry in the absence of blow drying or the like.

The hair cleaning composition may be applied from a container with an actuator that releases the compressed gas and the further components of the composition in a substantially consistent and uniform aerosol or spray. In one or more embodiments, the actuator may include a specialized valve, such as a Salvalco® Eco-Valve, or similar, to keep the discharge rate from the container consistent throughout the time of discharge of the hair cleaning composition from the container. The Salvalco® Eco-Valve, or similar, is an aerosol valve designed to use gases like nitrogen, carbon dioxide, or compressed air as propellants instead of LPG propellants while reducing or preventing clogging but still providing substantially consistent delivery of the composition from the container from use to use.

### EXAMPLES

The present disclosure is more fully illustrated by the following examples, which are set forth to illustrate certain embodiments of the present disclosure and are not to be construed as limiting thereof.

### Example 1: Alcohol and Disiloxane

First, alcohol (solvent) and conditioning agent (distearyldimonium chloride) were stirred together with hexamethyldisiloxane and surfactant (TEGO® Betaine F 50) in a beaker to form a liquid mixture. Next, in a 200 mL aluminum can the particulate cleaning agent (starch) and anti-caking agent (cetrimonium chloride) were added to the can along with a ball bearing. Then, the liquid mixture was added to the can while stirring. Then, a 170 mm Salvalco Eco-Valve® was added to the can and the can was crimped. The aluminum can was pressurized with 9 bar of nitrogen gas.

TABLE 1

| Raw Material | | | % active | Concentration | % active in |
|---|---|---|---|---|---|
| Trade Name | INCI name | Function | matter | (% w/w) | final product |
| Polygal RS UF | Oryza Sativa Starch | Absorbent | 99.9700 | 17.4500 | 17.4447650000 |
| | Cetrimonium Chloride | Anti-caking | 0.0300 | | 0.0052350000 |
| | | agent | | | |
| DEB 100 Alcohol | Alcohol Denat. | Solvent | 100.0000 | 51.9127 | 51.9127400000 |
| Varisoft ® TA | Distearyldimonium | Conditioning | 100.0000 | 0.0092 | 0.0091800000 |
| 100 | Chloride | agent | | | |
| Disiloxane | Hexamethyldisiloxane | Solvent | 100.0000 | 28.1000 | 28.1000000000 |
| TEGO ® Betaine | Cocamidopropyl | Surfactant | 100.0000 | 0.0600 | 0.0600000000 |
| F 50 | betaine | | | | |
| Nitrogen | Nitrogen | Propellant | 100.0000 | 2.4681 | 2.4680850000 |

### Example 2: Water, Alcohol and Disiloxane

As another example, water, alcohol, and disiloxane were used as the solvents. First, alcohol (solvent) and conditioning agent (distearyldimonium chloride) were stirred together with RO water, hexamethyldisiloxane, and surfactant (TEGO® Betaine F 50) in a beaker. Next, in a 200 mL aluminum can the absorbent material (starch) and anticaking agent (cetrimonium chloride) were added to the can along with a ball bearing. Then, the liquid mixture was added to the can while stirring. Then, a 170 mm Salvalco Eco-Valve® was added to the can and the can was crimped. The aluminum can was pressurized with 9 bar of nitrogen gas.

TABLE 2

| Raw Material | | | % active | Concentration | % active in |
|---|---|---|---|---|---|
| Trade Name | INCI name | Function | matter | (% w/w) | final product |
| Polygal RS UF | Oryza Sativa Starch | Absorbent | 99.9700 | 17.4500 | 17.4447650000 |
| | Cetrimonium | Anti-caking | 0.0300 | | 0.0052350000 |
| | Chloride | agent | | | |
| DEB 100 | Alcohol Denat. | Solvent | 100.0000 | 43.513 | 43.5127000000 |
| Alcohol | | | | | |
| Reverse | Aqua | Solvent | 100.0000 | 26.5000 | 26.5000000000 |
| Osmosis water | | | | | |
| Varisoft ® TA | Distearyldimonium | Conditioning | 100.0000 | 0.0092 | 0.0091800000 |
| 100 | Chloride | agent | | | |
| Disiloxane | Hexamethyldisiloxane | Solvent | 100.0000 | 10.0000 | 10.0000000000 |
| TEGO ® | Cocamidopropyl | Surfactant | 100.0000 | 0.0600 | 0.0600000000 |
| Betaine F 50 | betaine | | | | |
| Nitrogen | Nitrogen | Propellant | 100.0000 | 2.4681 | 2.4680850000 |

### Example 3: Alcohol and Disiloxane

As another example, only alcohol and disiloxane were used as the solvents. First, alcohol (solvent) and the conditioning agents were stirred together with hexamethyldisiloxane and surfactant in a beaker. Next, in a 200 mL aluminum can the absorbent material (starch) and anticaking agent (cetrimonium chloride) were added to the can along with a ball bearing. In this example, the absorbent material (starch) and anticaking agent (cetrimonium chloride) were added in a lower amount when compared with Examples 1 and 2. Then, the liquid mixture was added to the can while stirring. Then, a 170 mm Salvalco Eco-Valve® was added to the can and the can was crimped. The aluminum can was pressurized with 9 bar of nitrogen gas.

TABLE 3

| | | % | | | % active |
|---|---|---|---|---|---|
| Raw Material | | active | | Concentration | in final |
| Trade Name | INCI name | matter | Function | (% w/w) | product |
| Polygal RS UF | Oryza Sativa Starch | 99.9700 | Absorbent | 8.0 | 7.9976 |
| | Cetrimonium | 0.0300 | Anti-caking | | 0.0024 |
| | Chloride | | agent | | |
| DEB 100 Alcohol | Alcohol Denat. | 100.0000 | Solvent | 51.4219 | 51.4219 |
| Cocos Nucifera | Cocos Nucifera | 100.0000 | Conditioning | 0.0980 | 0.0980 |
| (Coconut) water | (Coconut) water | | agent | | |
| Leuconostoc/Radish | Leuconostoc/Radish | 100.0000 | Conditioning | 0.0020 | 0.0020 |
| Root Ferment | Root Ferment Filtrate | | agent | | |
| Filtrate | | | | | |
| Disiloxane | Hexamethyldisiloxane | 100.0000 | Solvent | 37.55 | 37.55 |
| | Lauryl betaine | 100.0000 | Surfactant | 0.06 | 0.06 |
| Nitrogen | Nitrogen | 100.0000 | Propellant | 2.4681 | 2.4681 |
| | Parfum | 100.0000 | Fragrance | 0.4000 | 0.4000 |

The formula of Example 3 was tested in a monadic study. The study included 38 subjects that were willing to participate in using the product at home. The subjects were instructed on usage of the product and used the product 2-3 times during a two-week trial period. At the end of the trial period, the subjects were asked to provide feedback regarding their experience.

The subjects reportedly had hair of different colors, thicknesses (i.e., fine, medium, or coarse), types (i.e., straight, wavy, or curly), lengths, and oily/dry texture.

Overall, a majority of the subjects were satisfied with the product containing the formula of Example 3 and found the product easy to use during the trial period. Those satisfied appreciated the fragrance, hair feel (i.e., refreshed, volumized, and less chalky than regular dry shampoo), and experienced less visible cloud or residue than their normal dry shampoo. The subjects also reported that they enjoyed the wet-to-dry format once they understood that the formula would apply wet but would dry quickly. The subjects reported that the fine mist was appreciated for an even and lighter coverage/application than traditional dry shampoos. The overall process was found simple and intuitive to subjects that have used spray dry shampoos before.

Only a few subjects reported feeling dissatisfied with the product, finding that the product performed inferior to expectations (i.e., less oil absorption, less volumizing effects, and less refreshed feeling), application experiences (i.e., unclear application and/or inconsistent spray), and/or disliked the fragrance.

The wetness of the product reportedly caused some difficulty separating sections of hair or applying as much product as the user normally would apply. The wetness of the product was brought up as a detractor for a handful of subjects, but it was not overwhelmingly mentioned, especially regarding overall satisfaction.

Of the 38 subjects, only three mentioned an alcohol odor, but one subject reported that the alcohol odor quickly dissipated.

About half of subjects stated they would likely use the product in the future. A few subjects indicated that they would use it regularly or switch completely to the product. Others found the usage to be situational, preferring for first day post-wash, non-sweaty days, on-the-go, or for light refreshes compared to their normal dry shampoo.

1. A hair cleaning composition, comprising:
a particulate cleaning agent in a total amount of about 2% to about 20% by weight, based on a total weight of the hair cleaning composition;
a plurality of solvents including at least one water-soluble or water-miscible solvent and at least one solvent that is not water-soluble or water-miscible, the plurality of solvents present in a total amount of about 70% to about 90% by weight, based on the total weight of the hair cleaning composition; and
a propellant consisting essentially of compressed gas, the propellant present in an amount of about 1% to about 5% by weight, based on the total weight of the hair cleaning composition,
wherein the hair cleaning composition leaves hair wet upon application to the hair and dries within a time of less than 30 seconds.

2. The hair cleaning composition of claim 1, wherein the at least one water-soluble or water-miscible solvent comprises an alcohol, water, or a mixture of the alcohol and water.

3. The hair cleaning composition of claim 1, wherein the at least one solvent that is not water-soluble or water-miscible comprises a siloxane.

4. The hair cleaning composition of claim 1, wherein the plurality of solvents comprises an alcohol, water, and a siloxane.

5. The hair cleaning composition of claim 1, wherein the plurality of solvents comprises an alcohol and a siloxane.

6. The hair cleaning composition of claim 3, wherein the siloxane comprises a disiloxane.

7. The hair cleaning composition of claim 6, wherein the disiloxane comprises hexamethyldisiloxane.

8. The hair cleaning composition of claim 1, wherein the particulate cleaning agent is present in an amount of about 8% to about 15% by weight based on the total weight of the hair cleaning composition.

9. The hair cleaning composition of claim 1, wherein the plurality of solvents is present in a total amount of about 75% to about 85% by weight, based on the total weight of the hair cleaning composition

10. The hair cleaning composition of claim 1, wherein the plurality of solvents is present in an amount of about 80% to about 90% by weight based on the total weight of the hair cleaning composition.

11. The hair cleaning composition of claim 1, wherein the compressed gas consists essentially of nitrogen gas.

12. The hair cleaning composition of claim 1, wherein the compressed gas consists of nitrogen gas.

13. The hair cleaning composition of claim 1, wherein the propellant is present in an amount of about 2.5% to about 3.5% by weight, based on the total weight of the hair cleaning composition.

14. The hair cleaning composition of claim 1, wherein the particulate cleaning agent comprises a starch.

15. The hair cleaning composition of claim 1, wherein the particulate cleaning agent comprises a rice starch.

16. The hair cleaning composition of claim 1, further comprising one or more materials selected from the group consisting of fragrances, anti-caking agents, conditioning agents, surfactants, preservatives, colorants, natural oils, or synthetic oils, and combinations thereof.

17. The hair cleaning composition of claim 1, configured to be applied to hair as an aerosol spray.

18. A method for cleaning hair, the method comprising the steps of:
spraying the hair with a hair cleaning composition delivered as an aerosol where the hair is wetted, the hair cleaning composition comprising:
a particulate cleaning agent;
a plurality of solvents; and
a propellant consisting essentially of nitrogen gas; and
mechanically dispersing the hair cleaning composition through the hair such that one or both of oil and grease on the hair is at least partially removed by the particulate cleaning agent and the wetted hair is substantially dry within a time of less than 30 seconds.

19. The method for cleaning hair of claim 18, wherein the step of mechanically dispersing the hair cleaning composition through the hair comprises brushing or combing.

20. The method for cleaning hair of claim 18, wherein the step of mechanically dispersing the hair cleaning composition through the hair comprises running of fingers through the hair.

21. The method for cleaning hair of claim 18, wherein the wetted hair is substantially dry within a time of about 2 seconds to about 20 seconds.

22. The method for cleaning hair of claim 18, wherein the wetted hair is substantially dry within a time of about 5 seconds to about 15 seconds.
